(19) United States
(12) Patent Application Publication
(10) Pub. No.: US 2005/0055100 A1
(43) Pub. Date: Mar. 10, 2005

(54) Total knee replacement for dogs

(76) Inventors: Ralph Harrison Lewis, Lakeport, CA (US); Toni Lewis, Lakeport, CA (US)

Correspondence Address: STRADLING YOCCA CARLSON & RAUTH;SUITE 1600, 660 NEWPORT CENTER DRIVE, P.O. BOX 7680, NEWPORT BEACH, CA, 92660, US

(21) Appl. No.: 10/657,763

(22) Filed: Sep. 8, 2003

(51) Int. Cl. A61F 2/38

(52) U.S. Cl. 623/20.28

(57) ABSTRACT

An unconstrained artificial knee joint for dogs is provided. A stainless steel femoral component has two condylar surfaces formed without any reinforcing ribs in order to minimize the amount of resection of the distal end of the femur. A femoral anchoring stem is formed integrally with the femoral component and is embedded in the femur. A metallic tibial support platform is provided which includes a tibial anchoring stem integrally formed with the platform, the stem adapted to extend downwardly into the tibia and be embedded in the tibia. A plastic spacer is carried by the upper surface of the tibial support and cooperates with and slides smoothly against the condylar surfaces of the femoral component.

### BACKGROUND AND BRIEF SUMMARY OF INVENTION

The present invention provides a full knee replacement specifically for dogs. More particularly, the present invention provides an unconstrained knee implant which minimizes the amount of the distal end of the femur that must be resected, which in turn creates a more solid anchorage for the implant and less pain for the animal.

Much of the prior art effort in designing artificial knee implants has been understandably concentrated on prosthetics for use in humans. The prior art has utilized dogs and other animals as test subjects with respect to various materials and designs for artificial knee implants. The present invention, in contrast, is intended solely for use in canines, both in respect to the materials used and in the design of the prosthesis, itself.

A typical prior art knee implant is shown in the Elias U.S. Pat. No. 5,480,443. The Elias patent teaches the use of a femoral component having a reinforcing portion 22 to which a femoral stem boss 24 is attached. The effect of utilizing extensive reinforcing sections is to require a relatively large portion of the distal end of the femur to be removed to attach the implant. Unfortunately, when a greater amount of the distal femur is removed, the anchoring stem inherently experiences a greater bending moment as bending stresses are experienced by the joint itself. By minimizing the amount of the femur that is resected, the anchoring stem is seated much closer to the working surfaces of the knee joint, and the bending stresses on the anchoring stem are reduced; therefore the prosthesis functions better. With reduced bending forces occurring between the anchoring stem and the femur, less pain is experienced by the animal and the prosthesis remains in place more securely and for a longer period of time.

The Amino et al U.S. Pat. No. 5,549,684 teaches an artificial knee joint wherein the femoral component is fabricated of ceramic material. The purpose of using ceramic material is to extend the useful life of the femoral component. A serious drawback of ceramic material is that it tends to be rather brittle and does not lend itself to the use of an integrally formed anchoring stem. Consequently, Amino requires the use of reinforcing ribs, which complicates the surgery of implanting the prosthetic and requires the resecting of more of the femur because of the presence of the ribs. The present invention, in its preferred embodiment, utilizes stainless steel with no reinforcing ribs for the femoral component as well as the tibial support platform. Stainless steel is much less brittle than ceramic and allows the use of an integrally formed anchoring stem. The present invention utilizes single anchoring stems in both the femoral and tibial components.

Another point of distinction between a prosthetic designed specifically for dogs and one designed for humans is that, in humans, 100% of the body weight is borne by the knees. In dogs, however, only 40% of the weight is borne by the knees. The reduction in percentage of body weight carried by the knees lessens the demand for a material capable of withstanding relatively large loads for a relatively long period of time. The use of stainless steel without reinforcing ribs, as taught by the present invention, allows for minimal resection of the distal end of the femur. Furthermore, the use of stainless steel provides a joint which has some “give” to it and which is better able to tolerate the shocks and impact loads experienced in canines.

The Jackson et al U.S. Pat. No. 4,034,418 teaches an artificial knee joint without any significant femoral anchor stem. The present invention provides a much more robust anchoring stem for both femoral and tibial components than Jackson.

It is accordingly a primary object of the present invention to provide an artificial knee joint designed specifically for dogs which minimizes the amount of resection of the distal end of the femur, which in turn reduces the stress between the femoral anchoring stem and the femur itself, resulting in a more solid anchorage for the implant and less pain for the animal.

A further object of the invention is to provide an unconstrained artificial knee implant for dogs wherein the shock and impact bearing surfaces are stainless steel bearing against plastic in order to increase the shock absorbing capacity of the implant when compared to implants utilizing ceramics.

Another object of the invention is to provide a canine knee replacement wherein a minimum amount of the distal femur is resected, thereby simplifying the surgical procedure in most instances.

Further objects and advantages will become apparent from the following description and drawings, wherein:

### BRIEF DESCRIPTION OF THE DRAWINGS

FIG. 1 is a perspective view of the three components of the present invention shown in exploded fashion;

FIG. 2 is a perspective view of the components illustrated in FIG. 1 wherein the spacer has been attached to the tibial support tray;

FIG. 3 is a schematic representation of the knee implant according to the present invention as applied to the distal end of the femur and the proximal end of the tibia; and

FIG. 4 is a rear view illustrating the invention as applied to the distal end of the femur and the proximal end of the tibia.

### DETAILED DESCRIPTION OF THE DRAWINGS

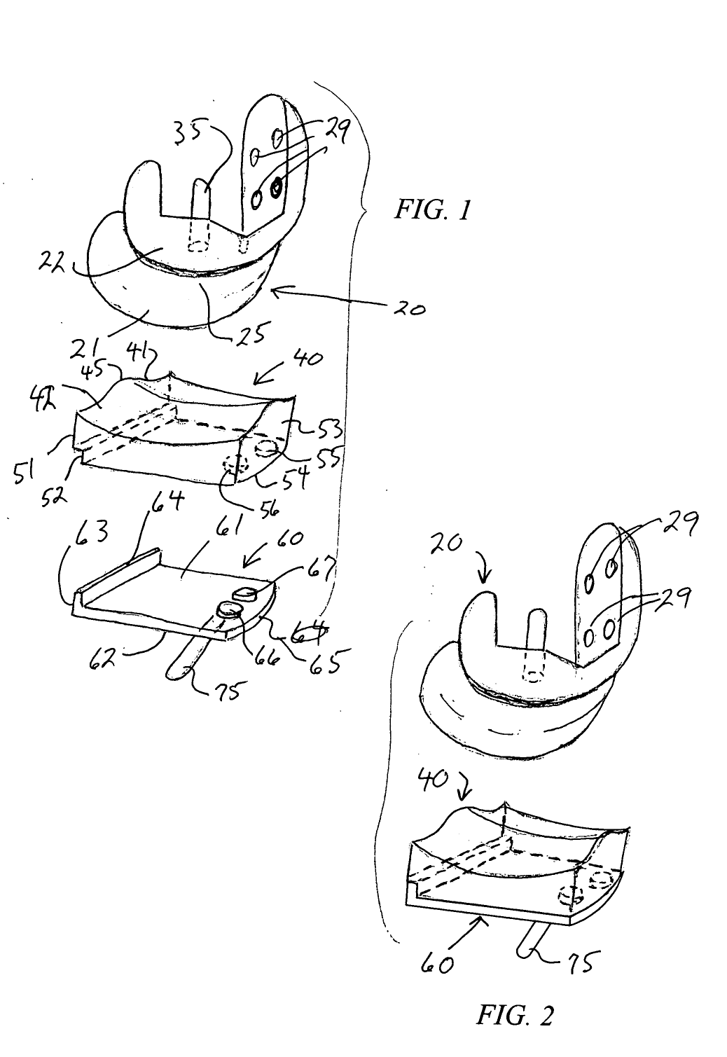

FIG. 1 illustrates in exploded form the three primary components of the present invention, namely, a femoral component 20, a tibial support 60 and a plastic spacer 40.

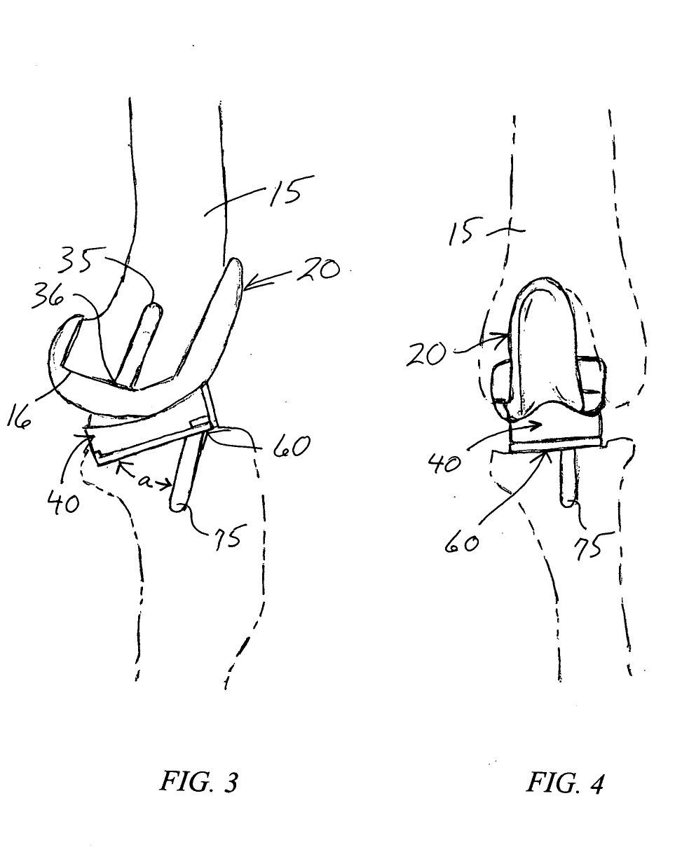

The femoral component 20 has two condylar surfaces 21 and 22 integrally formed preferably of a single piece of stainless steel. Between the two condylar surfaces 21 and 22 is a smooth recessed surface 25. A femoral anchoring stem 35 is provided which is embedded in femur 15 and serves to anchor the femoral component 20 to the femur 15 as shown in FIG. 3. The femoral component 20 and femoral anchoring stem 35 are formed as a single, monolithic piece of stainless steel. The femoral component 20 according to the present invention does not utilize reinforcing ribs, as noted above. By avoiding the use of reinforcing ribs, the femoral component 20 of the present invention minimizes the amount of the distal end of the femur that must be resected in order to implant the present invention. As shown in FIG. 3, the base 36 of anchoring stem 35 sits very close to the surface of plastic spacer 40. Base 36 of anchoring stem 35 is supported by the resected distal end 16 of femur 15. As noted above, by minimizing the amount of bone which must be removed or resected from the distal end of femur 15, the attachment of femoral component 20 to resected femur 15 is strengthened. For example, if reinforcing ribs having a thickness of one inch were added to femoral component 20, an additional one inch of bone would have to be removed from the distal end of femur 15. Removal of that additional material from the end of the femur would inherently cause significantly higher bending moments on anchoring stem 35 relative to femur 15 and would cause pain in the animal and/or loosening of the femoral component 20. Minimizing the amount of bone which must be removed from the distal end of the femur results in placement of the base 36 of anchoring stem 35 closer to the working surfaces of the joint, namely, the condylar surfaces 21 and 22 as well as the intermediate recess 25. The shape of condylar surfaces 21 and 22 mimics the shape of the head of a healthy canine femur. A plurality of depressioins 29 is provided in femoral component 20 to allow interdigitation of cement between the canine femur and the implant.

A metallic, preferably stainless steel, tibial support platform 60 has an upper surface 61 and a lower surface 62. A tibial anchoring stem 75 is carried by the lower surface 62 of tibial support platform 60. The stem 75 and platform 60 are preferably formed as a single, monolithic piece of stainless steel. The anchoring stem is adapted to extend downwardly into the tibia and be embedded in the tibia as illustrated in FIG. 3. As shown in FIG. 3, the tibial stem is oriented angularly at an angle “a” with respect to the lower surface 62 of approximately 60°. The tibial support platform has a first end 63 at which an upstanding edge 64 is formed. The upper surface 61 has a second end 65, near which two upstanding and generally cylindrical pegs 66 and 67 are formed. The purpose of the upstanding edge 64 and upstanding pegs 66 and 67 is to cooperate with recesses formed in plastic spacer 40 to mount spacer 40 securely to tibial support platform 60. A plastic spacer means 40 is made preferably of ultra high molecular weight plastic. The upper surface of spacer 40 includes a pair of shaped grooves 41 and 42 which are shaped to cooperate with and slide smoothly against condylar surfaces 21 and 22. A centrally disposed ridge 45 is designed to extend upwardly between condylar surfaces 21 and 22 and contact recess 25 so that, as femoral component 20 rotates with femur 15, there is a smooth sliding contact between the stainless steel femoral component 20 and the ultra high molecular weight spacer 40 carried by tibial support 60. Spacer 40 has a first end 51 in which an elongated recess 52 is formed. Recess 52 is formed with dimensions so that it fits snugly against upstanding edge 64. Spacer 40 has a second end 53 and a bottom surface 54. A pair of cylindrical recesses 55 and 56 is formed in bottom surface 54 near the second end 53 of spacer 40. Recesses 55 and 56 are designed to snugly snap onto upstanding pegs 56 and 57 to secure plastic spacer 40 to stainless steel tibial support 60.

The foregoing description of the invention has been presented for purposes of illustration and description and is not intended to be exhaustive or to limit the invention to the precise form disclosed. Modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is to be defined by the following claims.

1. An unconstrained artificial knee joint for dogs, adapted to be implanted at the distal end of the femur and proximal end of the tibia, comprising:
a metallic femoral component having two condylar surfaces integrally formed with a smooth recessed surface extending between said condylar surfaces, said femoral component being formed without any reinforcing ribs in order to minimize the resection of the distal femur,
a femoral anchoring stem carried by said femoral component, said anchoring stem adapted to extend upwardly into and be embedded in said femur,
a metallic tibial support platform having an upper surface and a lower surface,
a tibial anchoring stem carried by said lower surface of said tibial support platform, said anchoring stem adapted to extend downwardly into the tibia and be embedded in the tibia,
a plastic spacer means carried by said upper surface of said tibial support platform, said plastic spacer means being formed to cooperate with and slide smoothly against said condylar surfaces of said femoral component, and
mounting means for connecting said plastic spacer means to said tibial support platform.

2. The apparatus of claim 1 wherein said upper surface of said tibial support platform has first and second ends and said mounting means comprises an upstanding edge formed at said first end of the upper surface of said tibial support tray, one or more upstanding pegs formed adjacent said second end, and recesses formed in said spacer to engage said upstanding edge and said one or more upstanding pegs.

3. The apparatus of claim 1 wherein said femoral component and said femoral anchoring stem comprise a single, monolithic piece of stainless steel.

4. The apparatus of claim 2 wherein said tibial support tray and said tibial anchoring stem comprise a single, monolithic piece of stainless steel.

5. The apparatus of claim 1 wherein said plastic spacer means is made of ultra-high molecular weight plastic.
